(19) United States
(12) Patent Application Publication
(10) Pub. No.: US 2025/0276024 A1
(43) Pub. Date: Sep. 4, 2025

(54) A METHOD OF CELL REJUVENATION

(71) Applicant: Evonik Operations GmbH, Essen (DE)

(72) Inventors: Heike TOM DIECK, Friedrichsdorf (DE); Florian BÖHL, Neckargemünd (DE)

(73) Assignee: Evonik Operations GmbH, Essen (DE)

(21) Appl. No.: 18/861,952

(22) PCT Filed: Apr. 24, 2023
(86) PCT No.: PCT/EP2023/060576

§ 371 Date: Oct. 31, 2024

(30) Foreign Application Priority Data
May 4, 2022 (EP) 22171634.3

(51) Int. Cl. A61K 35/742; A61K 35/00; A61K 36/82; A61K 36/9066; A61K 38/05

(52) U.S. Cl. CPC A61K 35/742; A61K 36/82; A61K 36/9066; A61K 38/05; A61K 2035/115

(57) ABSTRACT

The present invention is related to composition for inducing rejuvenation of at least one cell, the composition comprising: —at least one probiotic Bacillus subtilis strain, and—at least one amino acid or peptide, wherein the amino acid is selected from glutamine, glutamic acid or salts thereof, and conjugated glutamine, and the peptide is an oligopeptide of 2-10 amino acid units in length, the amino acid units being natural amino acids, and at least one amino acid unit being a glutamine or glutamic acid unit.

### FIELD OF THE INVENTION

The present invention relates to a composition for inducing cell rejuvenation and/or decreasing the biological age of at least one cell or subject. The present invention also relates to a method of inducing cell rejuvenation and/or decreasing the biological age of a subject by administering the composition to the subject. In particular, the composition is a synbiotic composition comprising a probiotic Bacillus subtilis strain, and at least one amino acid.

### BACKGROUND OF THE INVENTION

Aging is known to involve gradual loss of function at the molecular, cellular, tissue and organismal levels. Aging of an individual and tissues, leads to the onset of various diseases. In particular, aged cells are frequently observed in inflammatory tissues in rheumatoid arthritis, osteoarthritis, hepatitis, chronic skin damage, arteriosclerosis, etc. When the aged cells accumulate, the damaged tissue cannot be recovered properly because the aged cells do not divide well.

At the chromatin level, aging is associated with the progressive accumulation of epigenetic errors that eventually lead to aberrant gene regulation, stem cell exhaustion, senescence, and deregulated cell/tissue homeostasis. In particular, Steve Horvath (2013) underpinned the role of epigenetics in senescence as he found that DNA methylation correlates with chronological age (Horvath S. (2013) Genome Biol.; 14(10): R115). Epigenetic age is highly correlated with the biological age and respond to environmental factors that accelerate or decelerate ageing processes, resulting in substantial deviations from the ‘real’ biological age. Hence, environmental confounders or cues contribute to either age acceleration or age deceleration whereas, age acceleration matches up with negative confounders/factors such as disease, stress, malnutrition, negative environmental conditions, etc. In contrast, both well-being and healthy nutrition tie in with age deceleration.

Epigenetic age acceleration (epigenetic age>chronological age) suggests that the underlying tissue ages faster than expected on the basis of chronological age, whereas a negative value (epigenetic age<chronological age, age deceleration) suggests that the tissue ages slower than would be expected. Epigenetic age acceleration is associated with a great number of age-related conditions and diseases, such as inflammatory processes.

As mentioned above, healthy nutrition is known to decrease epigenetic age. Accordingly, there is a need in the art for a simple and reliable food supplement that can regulate cellular senescence, and this can be used to prevent diseases in the long run. In particular, there is a need in the art for a food supplement that can be used to for rejuvenation of aged cells to young and healthy cells.

### DESCRIPTION OF THE INVENTION

The present invention attempts to solve the problems above by providing a food supplement that is capable of delaying cell senescence or returning cells to their original/earlier state and consequently decreasing the biological age of the cell or subject. Being a food supplement, it is not an essential component of the subject's diet. However, it has the advantage of decreasing the biological age of a cell or subject and/or inducing cell rejuvenation in the subject. Further, being a food supplement, it is also a very convenient way/tool that can be used to influence the biological age of a cell or subject in comparison to diet and/or physical activity. In particular, the food supplement is a synbiotic. More in particular, the food supplement is not only synbiotic (i.e. comprises a prebiotic and probiotic), but it also comprises other components that add to the benefits of the food supplement according to any aspect of the present invention.

As used herein the term ‘food supplements’ are defined to be concentrated sources of nutrients or other substances with a nutritional or physiological effect, whose purpose is to supplement the normal diet (www.efsa.europa.eu/en/topics/topic/food-supplements).

As used herein, the term ‘synbiotic’ refers to food ingredients or dietary supplements combining probiotics and prebiotics in a form of synergism. A synbiotic composition according to the present invention is therefore a composition comprising a probiotic strain and an amino acid, dipeptide, or oligopeptide as prebiotic. In particular, prebiotics and probiotics can be combined to support survival and metabolic activity of the latter and the resulting products belong to the class of synbiotics. An updated definition of the term prebiotics is: “a substrate that is selectively utilized by host microorganisms conferring a health benefit” (Gibson G R, Nat Rev Gastroenterol Hepatol 2017, 14(8):491-502.) thus referring not only to certain carbohydrates but also to e.g. amino acids and peptides such as dipeptides or oligopeptides. Prebiotics may also include other meaningful bioactive ingredients e.g. plant extracts, vitamins and the like.

According to one aspect of the present invention, there is provided a use of a composition for inducing rejuvenation of at least one cell, the composition comprising:
at least one probiotic Bacillus subtilis strain, and
at least one dipeptide selected from the group consisting of Glycine-Glutamine, Glycine-Glutamic acid, Alanine-Glutamine, Alanine-Glutamic acid and its acetylated forms.

As used herein, the phrase “rejuvenation of at least one cell” may mean restoring an aged cell to a young and healthy cell. In particular, “rejuvenated cell(s)” refers to aged cells that have been treated or transiently reprogrammed with one or more cellular reprogramming factors such that the cells have a transcriptomic profile of a younger cell while still retaining one or more cell identity markers.

As used herein, the term “cell” refers to an intact live cell, naturally occurring or modified. The cell may be isolated from other cells, mixed with other cells in a culture, or within a tissue (partial or intact), or an organism. In particular, the term “mammalian cell” refers to any cell derived from a mammalian subject. The cell may also be a cell derived from the culture and expansion of a cell obtained from a subject. The cell may also have been genetically modified to express a recombinant protein and/or nucleic acid. In particular, the mammalian cell may be a gut cell.

According to a further aspect of the present invention, there is provided a use of a composition for reducing the epigenetic age of at least one subject, the composition comprising:
at least one probiotic Bacillus subtilis strain, and
at least one dipeptide selected from the group consisting of Glycine-Glutamine, Glycine-Glutamic acid, Alanine-Glutamine, Alanine-Glutamic acid and its acetylated forms.

The composition according to any aspect of the present invention may comprise other components as well. These other components may include one or more plant extracts. In particular, the plant extracts may be selected from curcuma extract, green tea extract and combinations thereof. More in particular, the composition may also comprise other components such as Zinc, Vitamin B6, D-Biotin, Vitamin B12, Vitamin D, Pantothenic acid and the like. Even more in particular, the composition according to any aspect of the present invention comprises in addition to components (a) and (b), curcuma extract, green tea extract, Zinc, Vitamin B6, D-Biotin, Vitamin B12, Vitamin D, and Pantothenic acid.

The biological age depends on the biological state or condition of an individual or of a population and takes into account the circumstances of life (such as stress, nutrition, etc.). The terms “epigenetic age”, “methylation age”, and “biological age” have identical meanings and are used interchangeably in the context of the present application.

The term “chronological age” refers to the calendar time that has passed from birth/hatch. As used herein, the term “epigenetic clock” refers to a biochemical test that can estimate the DNA methylation age/epigenetic age in specific tissues or tissue-independently and can predict mortality and time left before death (Horvath 2013).

The composition according to any aspect of the present invention may result in post-translational modification (PTM) of histones which is a crucial step in epigenetic regulation of genes. PTM may then result in the change in expression of the genes.

The phrase “post-translational modification of histone” used interchangeably with the phrase ‘histone modification’ as used herein refers to a covalent modification of the N-terminal tail of a histone protein. Examples of histone modifications include, for example, methylation, acetylation, phosphorylation, ubiquitination, and ADP-ribosylation. Histone modifications and other epigenetic mechanisms are crucial for regulating gene activity and cellular processes. Different histone modifications regulate different processes, such as transcription, DNA replication, and DNA repair. Deregulation of any of these modifications can shift the balance of gene expression leading to aberrant epigenetic patterns and cellular abnormalities. In one example, the one or more of the histone modifications may be methylations. In particular, the methylation may occur at, for example, H3-K4, H3-K9, H4-K2O, H3-K27, H3-K36, and/or H3-K79.

The composition according to any aspect of the present invention may be used to change histone PTMs thus changing gene expression and be used for treatment of diseases related to histone PTMs.

As used herein, the terms “subject” and “individual” are used interchangeably and may be any living organism. For example, a subject according to any aspect of the present invention may be a plant or animal of any kind, preferably a rearing animal (or rearing stock) or livestock, which may be vertebrates or invertebrates. Typical examples of invertebrates that may be useful for being a subject according to any aspect of the present invention may be prawn or crabs such as the marbled crayfish. Typical examples of vertebrates that may be useful for being a subject according to any aspect of the present invention may be fish or land animals such as chicken or other livestock that may be cultured. The subject used herein may also refer to any vertebrate subject, including, without limitation, humans and other primates, including non-human primates such as chimpanzees and other apes and monkey species; farm animals such as cattle, sheep, pigs, goats and horses; domestic mammals such as dogs and cats; rodents such as mice, rats, rabbits, hamsters, and guinea pigs; birds, including domestic, wild and game birds such as chickens, turkeys and other gallinaceous birds, ducks, geese, and the like. In particular, the subject is a mammal. More in particular, the mammal is selected from the group consisting of a mouse, a rat, a guinea pig, a dog, a mini-pig, a human being, a cow, a sheep, a pig, a goat, a horse, a donkey, and a mule.

The probiotic strain in the composition according to any aspect of the present invention may be selected from Bacillus subtilis DSM 32315, Bacillus subtilis DSM 32540, Bacillus subtilis DSM 32592 and mixtures thereof. In particular, the probiotic strain may be Bacillus subtilis DSM 32315.

In the context of the present invention, the expression “natural amino acids” shall be understood to include both the L-form and the D-form of the known 20 amino acids. In particular, the L-form, of the amino acid may be used in the composition according to any aspect of the present invention. In one example, the term “amino acid” also includes analogues or derivatives of those amino acids.

As used herein, the term “free amino acid”, is understood as being an amino acid having its amino and its (alpha-) carboxylic functional group in free form, i.e., not covalently bound to other molecules, e.g., an amino acid not forming a peptide bond. Free amino acids may also be present as salts or in hydrate form. When referring to an amino acid as a part of, or in, an oligopeptide, this shall be understood as referring to that part of the respective oligopeptide structure derived from the respective amino acid, according to the known mechanisms of biochemistry and peptide biosynthesis.

A “peptide” shall be understood as being a molecule comprising at least two amino acids covalently coupled to each other by a peptide bond (R1-CO—NH—R2).

The amino acid or peptide in the composition according to any aspect of the present invention may be an oligopeptide with at least 2 amino acids. In particular, the oligopeptide may comprise alanine or glycine. More in particular, the oligopeptide may be selected from the group consisting of Glycine-Glutamine, Glycine-Glutamic acid, Alanine-Glutamine, Alanine-Glutamic acid and its acetylated forms.

The composition according to any aspect of the present invention may be in the form of a pill, capsule, tablet, powder blend, granules or liquid.

In one example, the composition according to any aspect of the present invention may be in the form of a capsule and the capsule comprises between 1×10⁸ and 2×10¹⁰ CFU of the probiotic strain and between 50 mg and 800 mg of the dipeptide.

In particular, the total amount of probiotic strain and amino acid or peptide is at 10 least 40 weight-%, particularly at least 50 weight-% more particularly at least 60 weight-%, even more particularly at least 70 weight-% of the total weight of the composition.

In particular, the total amount of plant extracts in the composition according to any aspect of the present invention is at least 10 weight-%, particularly at least 20 weight-% more particularly between 20 and 40 weight-% of the total weight of the composition.

In one example, the composition according to any aspect of the present invention comprises an enteric coating, wherein the enteric coating comprises one or more of the following: methyl acrylate-methacrylic acid copolymers, cellulose acetate phthalate (CAP), cellulose acetate succinate, Hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, zein and the like.

According to another aspect of the present invention, there is provided a composition for use in inducing rejuvenation of at least one cell, the composition comprising:
at least one probiotic Bacillus subtilis strain, and
at least one dipeptide selected from the group consisting of Glycine-Glutamine, Glycine-Glutamic acid, Alanine-Glutamine, Alanine-Glutamic acid and its acetylated forms.

The use according to any aspect of the present invention may be a non-therapeutic use, particularly, a non-therapeutic food use. In particular, the composition according to any aspect of the present invention may be a food supplement and not treated as a medicament.

According to another aspect of the present invention, there is provided a composition for use in reducing the epigenetic age of at least one subject, the composition comprising:
at least one probiotic Bacillus subtilis strain, and at least one dipeptide selected from the group consisting of Glycine-Glutamine, Glycine-Glutamic acid, Alanine-Glutamine, Alanine-Glutamic acid and its acetylated forms.

In one example, the composition may also be as an “accompanying therapy” or ‘adjuvant therapy’. In such a case, the composition according to any aspect of the present invention may be used for preventive, prophylactic and/or therapeutic use.

The composition according to any aspect of the present invention further comprises one or more plant extracts selected from curcuma extract and green tea extract.

According to a further aspect of the present invention, there is provided a method for reducing the epigenetic age of at least one subject, the method comprising administrating a composition comprising:
at least one probiotic Bacillus subtilis strain, and
at least one dipeptide selected from the group consisting of Glycine-Glutamine, Glycine-Glutamic acid, Alanine-Glutamine, Alanine-Glutamic acid and its acetylated forms
   to a subject in need thereof.

According to another aspect of the present invention, there is provided a method for inducing rejuvenation of at least one cell, the method comprising administrating a composition comprising:
at least one probiotic Bacillus subtilis strain, and
at least one dipeptide selected from the group consisting of Glycine-Glutamine, Glycine-Glutamic acid, Alanine-Glutamine, Alanine-Glutamic acid and its acetylated forms
   to a subject in need thereof.

The method according to any aspect of the present invention wherein the probiotic strain is selected from the group consisting of Bacillus subtilis DSM 32315, Bacillus subtilis DSM 32540, Bacillus subtilis DSM 32592 and mixtures thereof and/or the composition further comprises one or more plant extracts selected from curcuma extract and green tea extract.

A therapeutically effective dose or amount of the composition according to any aspect of the present invention may be administered to the subject in need. By “therapeutically effective dose or amount” is intended an amount of that is capable of inducing rejuvenation of at least one cell that may in the long run bring about a positive therapeutic response in the subject in need of tissue repair or regeneration, such as an amount that restores function and/or results in the generation of new tissue at a treatment site. Thus, for example, a “positive therapeutic response” would be an improvement in an age-related disease or condition in association with the therapy, and/or an improvement in one or more symptoms of the age-related disease or condition in association with the therapy, such as restored tissue functionality, reduced pain, improved stamina, increased strength, increased mobility, and/or improved cognitive function. The exact dose required will vary from subject to subject, depending on the species, age, and general condition of the subject, the severity of the condition being treated, mode of administration, and the like. An appropriate “effective” amount in any individual case may be determined by one of ordinary skill in the art using routine experimentation, based upon the information provided herein.

### BRIEF DESCRIPTION OF FIGURES

FIG. 1 is a graph showing the change in methylation of the 18 subjects which confirm the decrease in the biological age of the subjects after taking the food supplement according to any aspect of the present invention.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

A Proof-of-Concept Study to Evaluate the Impact of a Food Supplement (SAMANA® FORCE Comprising Bacillus subtilis DSM 32315, L-Alanyl-L-Glutamine, Curcuma Extract, Green Tea Extract, Zinc, Vitamin B6, D-Biotin, Vitamin B12, Vitamin D, Pantothenic Acid) on Production of Short Chain Fatty Acids and Microbiome in Healthy Subjects

Proof of concept study to investigate the impact of a food supplement Samana® Force consisting of a probiotic, dipeptide, plant extracts as well as vitamins and minerals on changes of gut microbiota and production of short chain fatty acids. Additionally, stool and blood biomarkers with respect to gut barrier and GI health were assessed. Furthermore, tolerability and parameters of bowel function (stool frequency, stool consistency and GI symptoms) were assessed during the study intervention.

### Test Product, Dose and Mode of Administration:

SAMANA® FORCE with 2 billion colony forming units of Bacillus subtilis DSM 32315, 290 mg L-Alanyl-L-Glutamine, 90 mg Curcuma extract (approx. 70-80% Curcumin), 90 mg, Green tea extract (approx. 50% EGCG), 5 mg Zinc, 0.56 mg Vitamin B6, 20 μg D-Biotin, 0.75 μg Vitamin B12, 4 μg Vitamin D, 2.4 mg Pantothenic acid

Mode of administration: 1 capsule in the morning (flexible with or without breakfast) and 1 capsule in the evening (flexible with or without dinner) unchewed with water

Duration of intervention: Two weeks run-in period, followed by a four-week intervention period and a two-week follow-up phase.

### Criteria for Evaluation:

The main focus of the study was to determine the effects of Samana® Force on the change in production of short chain fatty acids (SCFA) over a four-week intake phase in faeces and blood. Additionally, the gut microbiota assessed by 16S-rRNA was analysed.

### Statistical Methods:

The study was performed as exploratory proof of concept study. Changes over time were investigated. All data obtained in this study and documented in the eCRFs, questionnaires and diaries were listed and summarized with descriptive statistics or frequency tables as appropriate. All statistical tests were performed two-sided. A p-value less than 5% was considered a statistically significant. Bioinformatics was applied for microbiome analysis by a sequencing company.

### Results & Conclusions:

A significant increase of the SCFA butyrate was observed over the study period (Baseline vs. 2 weeks p=0.0278, Baseline vs. 4 weeks p=0.0728). To what extent this can be primarily attributed to the probiotic Bacillus subtilis and the metabolism of the provided dipeptide or if this results from further shifts in microbiota associated with an increase of amino acid converters cannot be derived from the data. However, data clearly indicate effects of the supplement on the microbiome which also resulted in changes of gut biomarkers but not in stool frequency and consistency. A significant increase of secretory IgA was identified (Baseline vs. 4 weeks p=0.0151 (n=17)), indicating immune-stimulating effects.

Possibly attributed to the rather unhealthy food habits characterized as low in fiber and low amounts of fruit and vegetables, rather high biomarkers of leaky gut and calprotectin were identified in the study collective despite no symptomatic clinical signs or history of any gastrointestinal disorders. Furthermore, a significant reduction on total (Baseline vs. 4 weeks p=0.0037) and LDL cholesterol (Baseline vs. 4 weeks p=0.0313) was observed. Possibly also attributed to the impact of microbiome or other direct mode of actions of the ingredients in SAMANA® FORCE, a highly significant decrease of fasting total GLP-1 and PYY was observed (Baseline vs. 4 weeks p<0.001).

### Conclusions:

In conclusion, over the intervention of 4 weeks with SAMANA® FORCE, the pilot study points to a significant increase of the SCFA butyrate in stool samples accompanied with interesting shifts in microbiome, with enhancing known butyrate-producer such as Faecalibacterium prausnitzii, as well as metabolic biomarkers and gut hormones.

### Example 2

### Difference in Biological Age (Start and End) Using Blood Samples

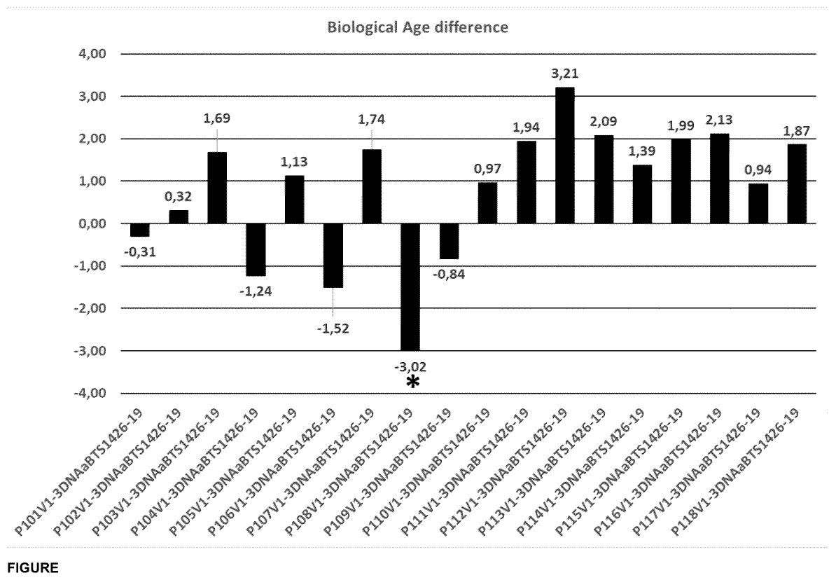

DNA was extracted from whole blood samples obtained from 18 subjects who were given SAMANA® FORCE as provided in Example 1. In particular, 18 male subjects with bad eating habits were included in the study. The methylation pattern of their blood cells was analysed before and after 4 weeks of supplementation with SAMANA FORCE. This resulted in 36 samples that have been analysed for this trial. Methylation analysis of the DNA was carried out by Elysium Health Inc. using the APEX platform that tests blood samples with the Horvath Clock. The results are shown in FIG. 1. Butyrate-forming synbiotic and additives like curcumin seem to show a trend towards age deceleration. For all 18 subjects the biological age was determined at two time points through their DNA methylation profile. The table 1 shows the biological age difference between V₁ (Start point) and V₃ (End point) for a subject: Age difference=V₁−V₃; If the value is positive, age is decelerating as V₁>V₃; if negative, age is accelerating, and the subject is older at V₃ than it was at the beginning of the study (V₁<V₃).*=Subject 108 had at time point V₃ fever and was invalidated.

TABLE 1
Biological age measured using Index, assigned genotype ids,
and predicted sex are shown for all samples that passed QC.

| | | | | Pre- |
|---|---|---|---|---|
| | | Biological | Biological | dicted |
| No. | Sample Name | Age at V1 | Age at V3 | Sex |
| 1 | P101V1DNAaBTS1426-19 | 24.82894396 | 25.13634675 | M |
| 2 | P110V1DNAaBTS1426-19 | 22.05914049 | 21.08967375 | M |
| 4 | P102V1DNAaBTS1426-19 | 19.15201899 | 18.83512651 | M |
| 5 | P111V1DNAaBTS1426-19 | 32.0491071 | 30.10470281 | M |
| 6 | P103V1DNAaBTS1426-19 | 33.20668112 | 31.51732682 | M |
| 7 | P112V1DNAaBTS1426-19 | 24.73127984 | 21.51909279 | M |
| 8 | P104V1DNAaBTS1426-19 | 21.18660006 | 22.42364451 | M |
| 9 | P113V1DNAaBTS1426-19 | 37.65507853 | 35.56900157 | M |
| 10 | P105V1DNAaBTS1426-19 | 28.93519845 | 27.80180738 | M |
| 11 | P114V1DNAaBTS1426-19 | 20.37200614 | 18.98630933 | M |
| 12 | P106V1DNAaBTS1426-19 | 24.4870228 | 26.00329832 | M |
| 13 | P115V1DNAaBTS1426-19 | 20.74961946 | 18.76426372 | M |
| 14 | P107V1DNAaBTS1426-19 | 30.27665755 | 28.53246647 | M |
| 15 | P116V1DNAaBTS1426-19 | 25.72510126 | 23.59694252 | M |
| 16 | P108V1DNAaBTS1426-19 | 26.09691986 | 29.11543572 | M |
| (*) | | | | |
| 17 | P117V1DNAaBTS1426-19 | 32.35184883 | 31.40850759 | M |
| 18 | P109V3DNAaBTS1426-19 | 14.99153386 | 15.82899323 | M |
| 19 | P118V3DNAaBTS1426-19 | 20.96980766 | 19.10397507 | M |

1. Use of a composition for inducing rejuvenation of at least one cell, the composition comprising:
at least one probiotic Bacillus subtilis strain, and
at least one dipeptide selected from the group consisting of Glycine-Glutamine, Glycine-Glutamic acid, Alanine-Glutamine, Alanine-Glutamic acid and its acetylated forms.

2. Use of a composition for reducing the epigenetic age of at least one subject, the composition comprising:
at least one probiotic Bacillus subtilis strain, and
at least one dipeptide selected from the group consisting of Glycine-Glutamine, Glycine-Glutamic acid, Alanine-Glutamine, Alanine-Glutamic acid and its acetylated forms.

3. Use according to claim 2, wherein the subject is a mammal, preferably the mammal is selected from the group consisting of selected from the group consisting of a mouse, a rat, a guinea pig, a dog, a mini-pig, a human being, a cow, a sheep, a pig, a goat, a horse, a donkey, and a mule.

4. Use according to claim 1, wherein the cell is a mammalian cell, preferably a human cell.

5. Use according to claim 1, wherein the composition further comprises one or more plant extracts selected from curcuma extract and green tea extract.

6. Use according to claim 1, wherein the probiotic strain is selected from the group consisting of Bacillus subtilis DSM 32315, Bacillus subtilis DSM 32540, Bacillus subtilis DSM 32592 and mixtures thereof.

7. Use according to claim 1, wherein the total amount of probiotic strain and dipeptide is at least 40 weight-% of the composition and/or the total amount of plant extract is at least 10 weight-% of the total weight of the composition.

8. Use according to claim 1, wherein the composition comprises an enteric coating, wherein the enteric coating comprises at least one of the following: methyl acrylate-methacrylic acid copolymers, cellulose acetate phthalate (CAP), cellulose acetate succinate, Hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate and zein.

9. A composition for use in inducing rejuvenation of at least one cell, the composition comprising:
at least one probiotic Bacillus subtilis strain, and
at least one dipeptide selected from the group consisting of Glycine-Glutamine, Glycine-Glutamic acid, Alanine-Glutamine, Alanine-Glutamic acid and its acetylated forms.

10. A composition for use in reducing the epigenetic age of at least one subject, the composition comprising:
at least one probiotic Bacillus subtilis strain, and
at least one dipeptide selected from the group consisting of Glycine-Glutamine, Glycine-Glutamic acid, Alanine-Glutamine, Alanine-Glutamic acid and its acetylated forms.

11. The composition according claim 9, wherein the composition further comprises one or more plant extracts selected from curcuma extract and green tea extract.

12. The composition according to claim 9, wherein the probiotic strain is selected from the group consisting of Bacillus subtilis DSM 32315, Bacillus subtilis DSM 32540, and Bacillus subtilis DSM 32592.

13. A method for reducing the epigenetic age of at least one subject, the method comprising administrating a composition comprising:
at least one probiotic Bacillus subtilis strain, and
at least one dipeptide selected from the group consisting of Glycine-Glutamine, Glycine-Glutamic acid, Alanine-Glutamine, Alanine-Glutamic acid and its acetylated forms to a subject in need thereof.

14. A method for inducing rejuvenation of at least one cell, the method comprising administrating a composition comprising:
at least one probiotic Bacillus subtilis strain, and
at least one dipeptide selected from the group consisting of Glycine-Glutamine, Glycine-Glutamic acid, Alanine-Glutamine, Alanine-Glutamic acid and its acetylated forms to a subject in need thereof.

15. The method according to claim 13, wherein the probiotic strain is selected from the group consisting of Bacillus subtilis DSM 32315, Bacillus subtilis DSM 32540, and Bacillus subtilis DSM 32592.

16. The method according to claim 13, wherein the composition further comprises one or more plant extracts selected from curcuma extract and green tea extract.
